(19) United States
(12) Patent Application Publication
(10) Pub. No.: US 2024/0197187 A1
(43) Pub. Date: Jun. 20, 2024

(54) Fever-Diagnostic Label and Fever-Diagnostic Mask

(71) Applicant: Try and E Co., Ltd., Kobe-shi, Hyogo (JP)

(72) Inventor: Takahiro Fujii, Kobe-shi (JP)

(21) Appl. No.: 17/625,098

(22) PCT Filed: Mar. 5, 2021
(86) PCT No.: PCT/JP2021/008604

§ 371 Date: Feb. 20, 2024

(51) Int. Cl. A61B 5/01; A41D 13/11; A61B 10/00

(52) U.S. Cl. CPC A61B 5/01; A41D 13/11; A61B 10/00; A61B 2010/0006

(57) ABSTRACT

Issue: An objective of the present invention is, without having the objective be measuring temperature, to make available at low cost a fever-diagnostic label as well a fever-diagnostic mask whereby fever state in a wearer may be diagnosed in a form in which it can be seen with the eyes.

Resolution Means: In order to achieve the just-noted objective, the present invention is characterized in providing a body-temperature thermochromic strip that is responsive to a wearer's body temperature, and an ambient thermochromic strip that is responsive to ambient temperature, and in displaying the body-temperature state of a wearer by comparing change in the body-temperature thermochromic strip responding to the wearer's body temperature with change in the ambient thermochromic strip responding to ambient temperature.

### TECHNICAL FIELD

The present invention relates to labels and surgical masks that by the act of being worn display the body-temperature state of the wearer, and to a fever-diagnosing method exploiting these.

With epidemics of influenza and the novel coronavirus and other infectious viral diseases having been threatening people's lives in recent years, in hospitals, schools, businesses, etc., diagnosing in some way, shape, or form the entry into the premises of an infected person who is running a fever is a crucial issue.

As conventional technology for diagnosing at a remove the presence of a person running a fever, examples that may be cited include the infrared thermographic device of Patent Document 1, and the “Digital Thermotape” of Non-Patent Document 1, utilizing a thermal indicator.

### PRECEDENT TECHNICAL LITERATURE

### Patent Documents

Patent Document 1: Japanese Pat. Pub. No. 5,399,737

### Non-Patent Documents

### Non-Patent Document 1:

https://www.nichigi.co.jp/products/samo/digital_thermotape.html

### SUMMARY

### Issues Claimed Subject Matter is to Address

Nevertheless, with its mechanics being complex, the infrared thermographic device involving Patent Document 1 proves to be costly for the user. The “Digital Thermotape” involving Non-Patent Document 1 is affixed directly to the skin, or is otherwise affixed onto a thin-fabric article such as a mask that fits close to the skin, to diagnose fever-incident states by gauging the appearance of a thermochromic strip on the tape that is responsive to the wearer's body temperature. While the tape is less expensive by far than the infrared thermographic device of Patent Document 1, the appearance-gauged temperature is not just body temperature; it is influenced considerably by the ambient air temperature. With the liquid crystals, thermochromic dyes, thermochromic pigments, etc. having been suited to the application beforehand, their response temperature is a set value, wherein depending on the ambient air temperature, there are instances in which while the wearer has a normal temperature (is someone in ordinary health) a color-change diagnosing incidence of fever arises in the thermochromic strip, or instances in which even with a person who has a fever, a color-change that does not diagnose incidence of fever arises along the thermochromic strip, such that correctly diagnosing incidence of fever is not possible.

An objective for the inventor in the present application is to make available a fever-diagnostic label that makes possible diagnosing incidence of fever more correctly, despite there being influence of ambient air temperature, than a fever-diagnostic label exploiting the properties of thermal indicators as is in Non-Patent Document 1.

### Ways of Addressing the Issues

In order to accomplish the just-stated objective, the present invention is characterized in being a fever-diagnostic label as well as a fever-diagnostic mask provided with a body-temperature thermochromic strip that is responsive to a wearer's body temperature, and an ambient thermochromic strip that is responsive to ambient temperature, for diagnosing the body-temperature state of the wearer by comparing color change in the body-temperature thermochromic strip with color change in the ambient thermochromic strip.

Here, the body-temperature thermochromic strip and the ambient thermochromic strip may be rendered such that they are formed by coating on ink or pigment that changes color depending on temperature, or else may be rendered such that they are formed by a sticker printed with the inks or pigments, or a thermochromic film employing heat-sensitive liquid crystals being fitted on.

Also, the mask body of the fever-diagnostic mask can be perforated with a desired one or plurality of holes in location(s) on the body-temperature thermochromic strip.

It is beneficial that a fever-diagnostic label as well as a fever-diagnostic mask involving the present invention be rendered with the ambient thermochromic strip segregated from the thermochromic strip on the fever-diagnostic label or fever-diagnostic mask, and the body-temperature thermochromic strip is furnished on the thermochromic strip on the fever-diagnostic label or fever-diagnostic mask, and with the ambient thermochromic strip furnished in a position where it may be sight-checked by an observer under the same ambient temperature as the wearer of the fever-diagnostic label or fever-diagnostic mask.

It is beneficial that the just-noted ambient thermochromic strip be rendered to furnish it with a temperature-gauging section for gauging the ambient temperature, an image-generating section for generating an image caused to produce color/change color according to the ambient temperature, and a display section for displaying an image output from the image-generating section. Also, implementations may be rendered so that on the just-noted body-temperature thermochromic strip and just-noted ambient thermochromic strip a labelling display is made for indicating the positions where color is produced/changed.

Further, the fever-diagnostic label can be employed affixed directly to the wearer's skin, and can also be employed affixed to a surgical mask or the like. What is more, implementations can be rendered in a configuration in which thermochromic ink is printed on a surgical mask or the like.

In addition, the present invention is a fever-diagnosing method wherein, in a fever-diagnostic label as well as a fever-diagnostic mask provided with a body-temperature thermochromic strip that is responsive to a wearer's body temperature and an ambient thermochromic strip that is responsive to ambient temperature, the body-temperature thermochromic strip and the ambient thermochromic strip are formed by coating on ink or pigment that changes color depending on temperature or else by fitting them on as a sticker, or otherwise are formed by fitting on a thermochromic film employing heat-sensitive liquid crystals, and the body-temperature state of the wearer is diagnosed by comparing color change in the body-temperature thermochromic strip with color change in the ambient thermochromic strip.

### Advantageous Effects of Invention

According to a fever-diagnostic label and a fever-diagnostic mask involving the present invention, comparing color change or display change in a body-temperature thermochromic strip that is responsive to body temperature, with color change or display change in an ambient thermochromic strip that is responsive to ambient temperature, enables visually and easily judging more accurately whether the body temperature of a person being inspected is higher than the body temperature (normal temperature) of someone in ordinary health, even with there being ambient temperature influences.

### BRIEF DESCRIPTION OF DRAWINGS

FIG. 1 is a diagram representing one example of a fever-diagnostic label involving the present invention (fever-diagnostic label: example that is stuck to the forehead).

FIG. 2 is a diagram representing one example in which color-producing/color-changing status that is observed in actuality has been schematically expressed.

FIG. 3 is a diagram representing a separate example in which color-producing/color-changing status that is observed in actuality has been schematically expressed.

FIG. 4 is a diagram representing one example in which the body-temperature thermochromic strip and the ambient thermochromic strip have been arranged in a circular form.

FIG. 5 is a diagram illustrating parts that support the ambient thermochromic strip having been provided.

FIG. 6 is a diagram representing one example of a fever-diagnostic mask involving the present invention (fever-diagnostic mask: example that is furnished on the breathing area).

FIG. 7 is a diagram representing a separate example of a fever-diagnostic mask involving the present invention (fever-diagnostic mask: example that is furnished on an area that fits snugly against the skin).

FIG. 8 is a diagram representing a separate example of a fever-diagnostic mask involving the present invention (fever-diagnostic mask: example that is furnished on an area that fits snugly against the skin).

FIG. 9 is a diagram representing a separate mode of a fever-diagnostic label involving the present invention.

FIG. 10 is a diagram representing a separate mode of a fever-diagnostic label involving the present invention.

FIG. 11 is a diagram representing a separate mode of a fever-diagnostic label involving the present invention.

FIG. 12 is one example in which in Embodiment Example 5 color-producing/color-changing status that is observed in actuality has been schematically expressed.

FIG. 13 is a separate example in which in Embodiment Example 5 color-producing/color-changing status that is observed in actuality has been schematically expressed.

FIG. 14 is a further separate example in which in Embodiment Example 5 color-producing/color-changing status that is observed in actuality has been schematically expressed.

### BEST MODE FOR EMBODYING INVENTION

Hereinafter, based on the drawings, a description in detail of embodiment examples of the present invention will be made. In the drawings, identical parts are labeled with identical numbers; description that would be redundant is omitted. Also, it should be borne in mind that in some cases the drawings may express the present invention exaggeratedly for the sake of its understanding, wherein they are not necessarily making representations in fine detail. It should be understood that the technical scope of the present invention is not limited to the specific applications or forms/dimensions, etc. presented in the following embodying modes. Below, referring to the drawings an explanation of modes of embodying the present invention will be made.

### Embodiment Example 1

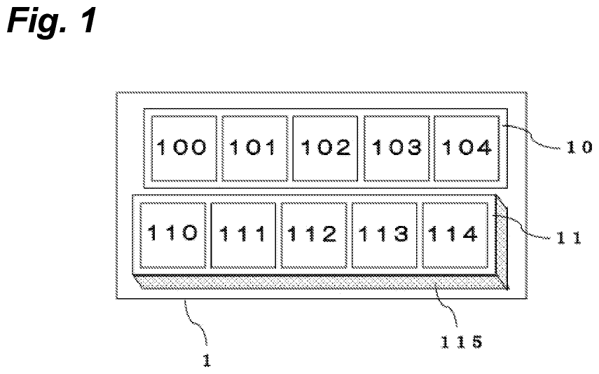

While referring to FIG. 1 through 5, a description of Embodiment Example 1 will be made. FIG. 1 is a diagram illustrating one example of a fever-diagnostic label involving the present invention (fever-diagnostic label: example that is stuck to the forehead). Affixing to a wearer's forehead the fever-diagnostic label 1 in FIG. 1 will diagnose whether or not the wearer is running a fever. In Embodiment Example 1, an instance of affixing to the forehead is being given, but the label can be affixed to an exposed area of the skin other than the forehead, including a cheek or arm.

Reference is made to FIG. 1. A fever-diagnostic label 1 involving the present invention is constituted by a body-temperature thermochromic strip 10 that produces color/changes color in response to body temperature, and an ambient thermochromic strip 11 that produces color/changes color in response to ambient temperature. The fever-diagnostic label 1 is affixed to the forehead of a wearer.

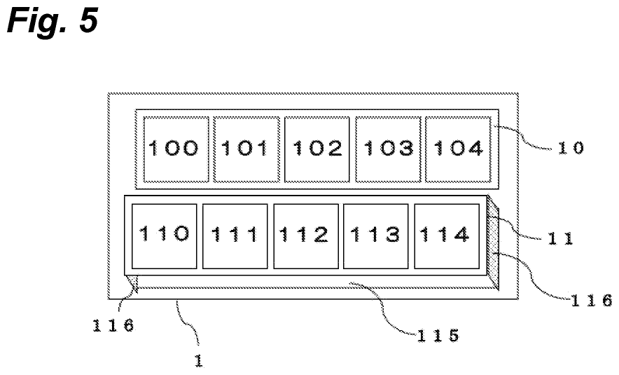

In order to lessen to the extent possible the impact from body temperature, rendering the ambient thermochromic strip 11 section to be furnished with a heat insulator 115 between itself and the wearer is ideal. The heat insulator 115 may be a solid substance such as polystyrene foam, or may be a layer of air the formation of which has been devised so as to provide it at a remove from the wearer. Here, parts 116 in FIG. 5 that support the ambient thermochromic strip 11 are provided, thereby having the heat insulator 115 be a layer of air.

On the body-temperature thermochromic strip 10, 100 through 104, and on the ambient thermochromic strip 11, 110 through 114, are each constituted by a thermal indicator that produces color/changes color according to temperature. This is made possible by, for example, utilizing cholesteric liquid crystals. Compounding a plurality of cholesterol-derivative substances whose melting points differ makes it possible to design them to exhibit color production in/color change to red, green, or blue in desired temperature ranges within an ordinary temperature range. Outside the designed temperature range, they are caused to color-change to black.

Utilizing a substance such as will exhibit color production in/color change to red, green, or blue at a 2° C. temperature spread, 100 to 104 on the body-temperature thermochromic strip 10 are each constituted to exhibit color production in/color change to red, green, or blue in temperature ranges of 25° C. to 27° C., 27° C. to 29° C., 29° C. to 31° C., 31° C. to 33° C., and 33° C. to 35° C. For example, in an instance in which the surface temperature of the forehead of the wearer of a fever-diagnostic label 1 is 30° C., the 102 section in FIG. 1 turns green (apart from it, 100, 101, 103 and 104 turn black). The temperature ranges herein should be adjusted to forehead surface-temperatures that can be observed; they are not limited to the specific examples given here.

On the ambient thermochromic strip 11, 110 to 114 are constituted as follows. Along 100 to 104 on the body-temperature thermochromic strip 10, just below the section where the surface temperature of a healthy person's forehead will produce color in/change color to green, disposed is a cholesteric liquid crystal whose temperature range is designed so that at the temperature of the environs in that situation, it will exhibit color production in/color change to green. For example, in instances in which it has been observed that the surface temperature of a healthy person's forehead is 26° C. when the ambient temperature is 12° C., that the surface temperature of a healthy person's forehead is 28° C. when the ambient temperature is 18° C., that the surface temperature of a healthy person's forehead is 30° C. when the ambient temperature is 24° C., that the surface temperature of a healthy person's forehead is 32° C. when the ambient temperature is 30° C., and that the surface temperature of a healthy person's forehead is 34° C. when the ambient temperature is 36° C., 110 to 114 on the ambient thermochromic strip 11 are arranged to exhibit color production in/color change to red, green, or blue in temperature ranges (6° C. temperature spreads) of 8° C. to 14° C., 14° C. to 20° C., 20° C. to 26° C., 26° C. to 32° C., and 32° C. to 38° C. This means, in other words, that it is designed so that when the fever-diagnostic label 1 is observed in a (healthy-person) instance in which the wearer has a normal temperature, the producing-color/changing-color state of the body-temperature thermochromic strip 10 and the producing-color/changing-color state of the ambient thermochromic strip 11 will be observed to be roughly the same.

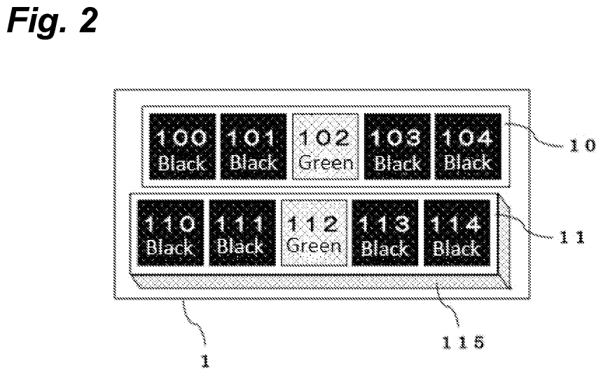

FIG. 2 is one example in which color-producing/color-changing status that is observed in actuality has been schematically expressed. In this case, because the producing-color/changing-color state of the body-temperature thermochromic strip 10 and the producing-color/changing-color state of the ambient thermochromic strip 11 are about the same, the wearer can be diagnosed as not having a fever. Specifically, with it being the case that an instance in which the ambient temperature is 24° C. and the surface temperature of the wearer's forehead is 30° C. is presented, because the strips have been designed, as the result of observation in advance, to cover an instance in which “the surface temperature of a healthy person's forehead is 30° C. when the ambient temperature is 24° C.,” the instance in FIG. 2 is an example in which the body-temperature state of a healthy person is displayed.

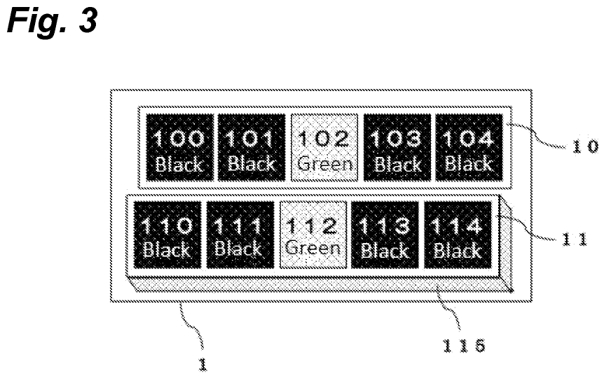

FIG. 3 is a separate example in which color-producing/color-changing status that is observed in actuality has been schematically expressed.

In this case, because the location in the body-temperature thermochromic strip 10 where producing-color/changing-color is exhibited is an area where the temperature is higher than (rightward of) the location in the ambient thermochromic strip 11 where producing-color/changing-color is exhibited, the wearer can be diagnosed as having a fever. Specifically, with it being the case that an instance in which the ambient temperature is 24° C. and the surface temperature of the wearer's forehead is 32° C. is presented, because the strips have been designed, as the result of observation in advance, to cover an instance in which “the surface temperature of a healthy person's forehead is 30° C. when the ambient temperature is 24° C.,” the instance in FIG. 3 is an example in which, with the surface temperature of the wearer's forehead being observed to be 2° C. higher than a healthy person's, the body-temperature state of a person having a fever is displayed.

In Embodiment Example 1 as the thermal indicator cholesteric liquid crystals were taken up, but as long as it is something that will produce color/change color according to designated temperature it can be adopted.

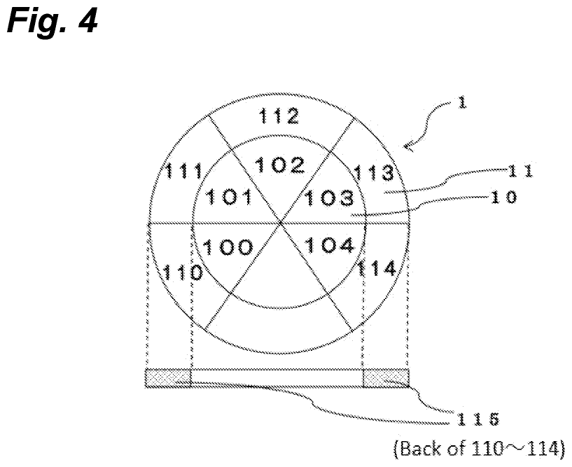

Furthermore, with the fever-diagnostic label 1, the body-temperature thermochromic strip 10 and the ambient thermochromic strip 11 have been landscape-oriented and arranged one above the other, but as in FIG. 4, arranging them in circular form is possible. Between 110 to 114 of the ambient thermochromic strip 11 and a mask body, a heat insulator 115 is provided, thereby enabling the body-temperature state of a wearer to be diagnosed more accurately. In the same way as in FIG. 5, by creating a structure with plastic or other polymer resin, the heat insulator 115 may be an air gap. Moreover, the upper/lower relationship between the body-temperature thermochromic strip 10 and the ambient thermochromic strip 11 in FIG. 1 can be reversed, or which between the body-temperature thermochromic strip 10 and the ambient thermochromic strip 11 in FIG. 4 is the inner-side/outer-side can be reversed.

### Embodiment Example 2

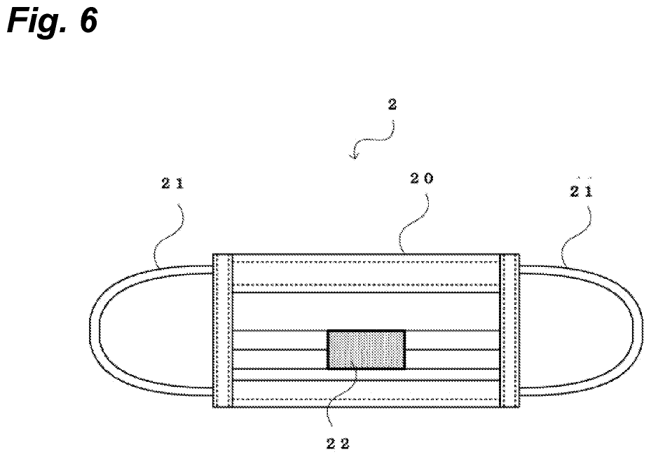

While referring to FIG. 6, a description of Embodiment Example 2 will be made. FIG. 6 is a diagram representing one example of a fever-diagnostic mask involving the present invention (fever-diagnostic mask: example that is furnished on the breathing area).

Reference is made to FIG. 6. A fever-diagnostic mask 2 involving the present invention is furnished with a mask body 20, constituted from a flexible material, having size enabling it to cover at least the wearer's nostrils and mouth, and a pair of elastic support cords 21, having stretchability, respectively attached to either lateral-edge portion of the mask body, and either ends of each of which are fixed to vertically differing locations on the lateral edge portions. Therein, on the outside surface of the mask body 20 of the fever-diagnostic mask 2 in or nearby the middle, a thermochromic strip 22 is provided.

Thanks to the thermochromic strip 22 being provided on the outside surface of the mask body 20 of the fever-diagnostic mask 2 in or nearby the middle, should the wearer's breathing be whichever, by nasal respiration or mouth respiration, the wearer's expiratory air will pass to the thermochromic strip 22, and due to color change in this thermochromic strip 22 having reacted to the expiratory air, the body-temperature state of the wearer will be displayed.

The thermochromic strip 22, being of the same configuration as that of the fever-diagnostic label 1 in Embodiment Example 1, can be represented as in FIG. 1. The body-temperature thermochromic strip 10 was in Embodiment Example 1 for observing the surface temperature of the wearer's forehead, whereas in Embodiment Example 2, it is for observing the temperature of the wearer's expiratory air. For example, in instances in which it has been observed that a healthy person's expiratory air temperature is 22° C. when the ambient temperature is 12° C., that a healthy person's expiratory air temperature is 24° C. when the ambient temperature is 18° C., that a healthy person's expiratory air temperature is 26° C. when the ambient temperature is 24° C., that a healthy person's expiratory air temperature is 28° C. when the ambient temperature is 30° C., and that a healthy person's expiratory air temperature is 30° C. when the ambient temperature is 36° C., 100 to 104 on the body-temperature thermochromic strip 10 are configured to exhibit color production in/color change to red, green, or blue in temperature ranges of, respectively, 21° C. to 23° C., 23° C. to 25° C., 25° C. to 27° C., 27° C. to 29° C., and 29° C. to 31° C., and 110 to 114 of the ambient thermochromic strip 11 are arranged to exhibit color production in/color change to red, green, or blue in temperature ranges (6° C. temperature spreads) of 8° C. to 14° C., 14° C. to 20° C., 20° C. to 26° C., 26° C. to 32° C., and 32° C. to 38° C.

Here, the area on the mask body 20 of the fever-diagnostic mask 2 where the thermochromic strip 22 is provided may be perforated with a desired one or plurality of holes, and thermochromic film(s), or sticker(s) printed with ink or pigment, may be fitted on in a manner so as to cover the hole(s), in such a way as to form 100 to 104 of the body-temperature thermochromic strip 10. Providing this sort of hole(s) is in order to facilitate the strip's being responsive to the temperature of the wearer's expiratory air.

Furthermore, in the same way as with Embodiment Example 1, the body-temperature thermochromic strip 10 and the ambient thermo-thermic strip 11 have been landscape-oriented and arranged one above the other, but as in FIG. 4, arranging them in circular form is possible. Between 110 to 114 of the ambient thermochromic strip 11 and the mask body, a heat insulator (which may be an air gap) is furnished, thereby enabling the body-temperature state of a wearer to be diagnosed more accurately. Moreover, likewise as with Embodiment Example 1, the upper/lower relationship between the body-temperature thermochromic strip 10 and the ambient thermochromic strip 11 in FIG. 1 can be reversed, or which between the body-temperature thermochromic strip 10 and the ambient thermochromic strip 11 in FIG. 4 is the inner-side/outer-side can be reversed.

In Embodiment Example 2, likewise as with Embodiment Example 1, as the thermal indicator cholesteric liquid crystals were taken up, but as long as it is something that will produce color/change color according to designated temperature it can be adopted. Depending on the thermal indicator to be adopted, it can be configured by printing directly onto the mask or else by sticking on a fever-diagnostic label in sticker form.

### Embodiment Example 3

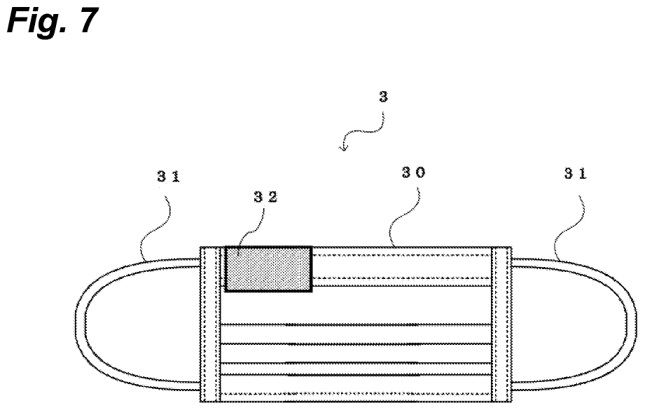

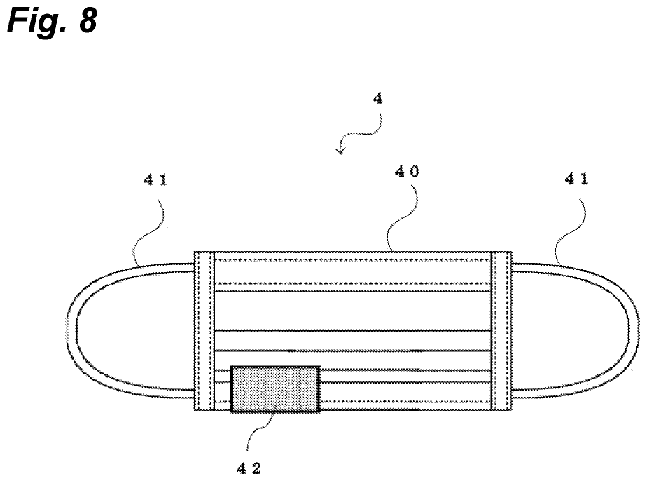

While referring to FIGS. 7 and 8, a description of Embodiment Example 3 will be made. FIGS. 7 and 8 each are a diagram representing a separate example of a fever-diagnostic mask involving the present invention (fever-diagnostic mask 3: example that is furnished on an area that fits snugly against the skin). In Embodiment Example 2, the thermochromic strip 22 was configured so as to be responsive to expiratory air, whereas in Embodiment Example 3, a thermochromic strip 32 (or 42) is furnished on an area along the mask body that fits relatively snugly against the skin, whereby it is configured so as to be responsive to skin-surface temperature, rather than to be responsive to expiratory air.

The areas along the mask body that fit relatively snugly against the skin are the upper cheek areas or the lower chin area. Furthermore, with not only the mask body but also the support-cord 31 (or 41) pieces being parts that fit snugly against the skin, furnishing on the support cords 31 (or 41) a thermochromic strip 32 (or 42) made into a sticker or made into a film is possible.

Reference is made to FIG. 7. A fever-diagnostic mask 3 involving the present invention is furnished with a mask body 30, constituted from a flexible material, having size enabling it to cover at least the wearer's nostrils and mouth, and a pair of elastic support cords 31, having stretchability, respectively attached to either lateral-edge portion of the mask body, and either ends of each of which are fixed to vertically differing locations on the lateral edge portions. Therein, on the upper-right edge of the outside surface of the mask body 30 of the fever-diagnostic mask 3, a thermochromic strip 32 is provided. On the outside surface of the mask body 20 of the fever-diagnostic mask 2 in or nearby the middle, a thermochromic strip 22 is provided. While the instance in FIG. 7 is on the upper-right edge, it is also embodiable on the upper-left edge.

The thermochromic strip 32 is of the same configuration as the fever-diagnostic label 1 of Embodiment Example 1, and can be represented as in FIG. 1.

Reference is made to FIG. 8. A fever-diagnostic mask 4 involving the present invention is furnished with a mask body 40, constituted from a flexible material, having size enabling it to cover at least the wearer's nostrils and mouth, and a pair of elastic support cords 41, having stretchability, respectively attached to either lateral-edge portion of the mask body, and either ends of each of which are fixed to vertically differing locations on the lateral edge portions. Therein, on the lower-right edge of the outside surface of the mask body 40 of the fever-diagnostic mask 4, a thermochromic strip 42 is provided. While in the instance in FIG. 8 the thermochromic strip 42 is on the lower-right edge, it is also embodiable on the lower-left edge.

The thermochromic strip 42 is of the same configuration as the fever-diagnostic label 1 of Embodiment Example 1, and can be represented as in FIG. 1.

Here, the area on the mask body 30 (or 40) of the fever-diagnostic mask 3 (or 4) where the thermochromic strip 32 (or 42) is provided may be perforated with a desired one or plurality of holes, and thermochromic film(s), or sticker(s) printed with ink or pigment, may be fitted on in a manner so as to cover the hole(s), in such a way as to form 100 to 104 of the body-temperature thermochromic strip 10. Providing this sort of hole(s) is in order to facilitate the strip's being responsive to the wearer's skin-surface temperature.

Furthermore, in the same way as with Embodiment Example 2, the body-temperature thermochromic strip 10 and the ambient thermo-thermic strip 11 have been landscape-oriented and arranged one above the other, but as in FIG. 4, arranging them in circular form is possible. Between 110 to 114 of the ambient thermochromic strip 11 and the mask body, a heat insulator (which may be an air gap) is furnished, thereby enabling the body-temperature state of a wearer to be diagnosed more accurately. Moreover, likewise as with Embodiment Example 2, the upper/lower relationship between the body-temperature thermochromic strip 10 and the ambient thermochromic strip 11 in FIG. 1 can be reversed, or which between the body-temperature thermochromic strip 10 and the ambient thermochromic strip 11 in FIG. 4 is the inner-side/outer-side can be reversed.

In Embodiment Example 3, likewise as with Embodiment Example 2, as the thermal indicator cholesteric liquid crystals were taken up, but as long as it is something that will produce color/change color according to designated temperature it can be adopted. Depending on the thermal indicator to be adopted, it can be configured by printing directly onto the mask or else by sticking on a fever-diagnostic label in sticker form.

### Embodiment Example 4

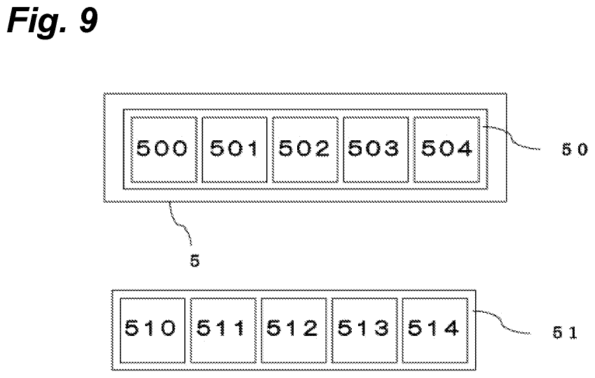

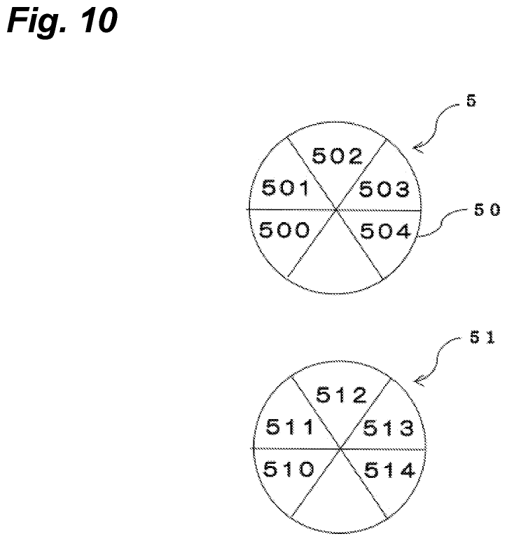

While referring to FIGS. 9 and 10, a description of Embodiment Example 4 will be made. The fever-diagnostic label 1 of Embodiment Example 1 and the thermochromic strip 22 or 32 (or 42) of Embodiment Example 2 or 3 were configured such as to be furnished with both the body-temperature thermochromic strip 10 responsive to expiratory air or surface temperature, and the ambient thermochromic strip 11 responsive to ambient temperature, whereas with Embodiment Example 4, as in FIG. 9, an ambient thermochromic strip 51 responsive to ambient temperature is segregated, and is arranged in an area that is the same ambient temperature as that of the wearer and where it may be sight-checked by an observer. Since the expiratory air and the skin-surface temperature of the wearer do not exert influence, a heat insulator underlying the mask body as in Examples 1 to 3 can be omitted.

In Embodiment Example 4, likewise as with Embodiment Example 1, a fever-diagnostic label will be described, but it may be implemented for a fever-diagnostic mask as in Embodiment Example 2 or 3.

A fever-diagnostic label 5 of Embodiment Example 4 will be described using FIG. 9. By affixing the fever-diagnostic label 5 in FIG. 9 to a wearer's forehead, fever diagnosis is carried out.

For example, in instance in which it has been observed that the surface temperature of a healthy person's forehead is 26° C. when the ambient temperature is 12° C., that the surface temperature of a healthy person's forehead is 28° C. when the ambient temperature is 18° C., that the surface temperature of a healthy person's forehead is 30° C. when the ambient temperature is 24° C., that the surface temperature of a healthy person's forehead is 32° C. when the ambient temperature is 30° C., and that the surface temperature of a healthy person's forehead is 34° C. when the ambient temperature is 36° C., 500 to 504 on the body-temperature thermochromic strip 50 are configured to exhibit color production in/color change to red, green, or blue in temperature ranges of 25° C. to 27° C., 27° C. to 29° C., 29° C. to 31° C., 31° C. to 33° C., and 33° C. to 35° C., respectively, and 510 to 514 on the ambient thermochromic strip 51 are arranged to exhibit color production in/color change to red, green, or blue in temperature ranges (6° C. temperature spreads) of 8° C. to 14° C., 14° C. to 20° C., 20° C. to 26° C., 26° C. to 32° C., and 32° C. to 38° C. The temperature ranges herein should be adjusted to forehead surface-temperatures that can be observed; they are not limited to the specific examples given here.

In Embodiment Example 4, when the body-temperature thermochromic strip 50 of the fever-diagnostic label 5 and the segregated ambient thermochromic strip 51 responsive to ambient temperature are observed in a (healthy-person) instance in which the wearer has a normal temperature, the producing-color/changing-color state of the body-temperature thermo-thermic strip 50 and the producing-color/changing-color state of the ambient thermochromic strip 51 will be observed as being roughly the same. In an instance in which the wearer has a fever, because the location in the body-temperature thermochromic strip 50 where producing-color/changing-color is exhibited is an area where the temperature is higher than (rightward of) the location in the ambient thermochromic strip 51 where producing-color/changing-color is exhibited, he/she can be diagnosed as someone with a fever.

In Embodiment Example 4, as the thermal indicator cholesteric liquid crystals were taken up, but as long as it is something that will produce color/change color according to designated temperature it can be adopted.

Furthermore, likewise as with Embodiment Example 1, the body-temperature thermochromic strip 50 and the ambient thermochromic strip 51 have been landscape-oriented, but as in FIG. 10, arranging them in circular form is possible.

### Embodiment Example 5

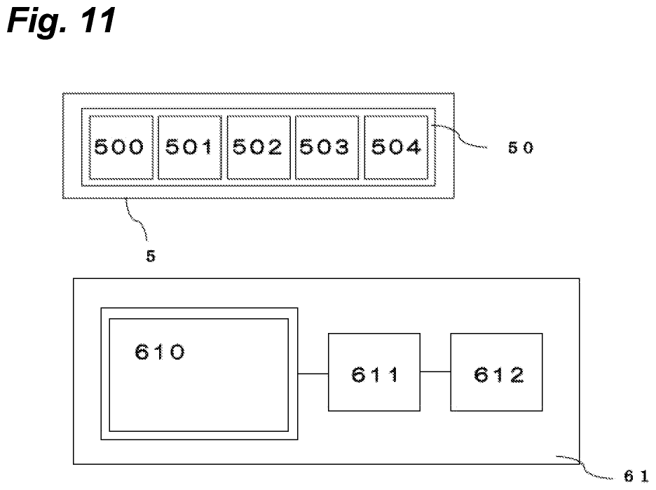

While referring to FIGS. 11, 12, 13, and 14, a description of Embodiment Example 5 will be made. With Embodiment Example 4, the body-temperature thermochromic strip 50 responsive to expiratory air or surface temperature, and the ambient thermochromic strip 51 responsive to ambient temperature are segregated and are arranged in areas where the ambient temperature is the same as that of the wearer and where they may be sight-checked by an observer, whereby a heat insulator underlying the mask body can be omitted. Here, the ambient thermochromic strip 51 responsive to ambient temperature can be embodied in such a way that, without utilizing a thermal indicator as in Embodiment Examples 1 to 4, it displays the equivalent information (images).

In Embodiment Example 5, likewise as with Embodiment Example 1, a fever-diagnostic label will be described, but it may be implemented for a fever-diagnostic mask as in Embodiment Example 2 or 3.

A fever-diagnostic label 5 of Embodiment Example 5 will be described using FIG. 11. The fever-diagnostic label 5 in FIG. 11 has the same configuration as the fever-diagnostic label 5 in FIG. 9 in Embodiment Example 4. By affixing the fever-diagnostic label 5 in FIG. 11 to a wearer's forehead, fever diagnosis is carried out. In Embodiment Example 5, the ambient thermochromic strip 61 of FIG. 11 is utilized instead of the ambient thermochromic strip 51 of FIG. 9. The ambient thermochromic strip 61 is constituted from a display section 610, an image-generating section 611, and a temperature-gauging section 612.

Supposed are exemplary instances in which it is observed that the surface temperature of a healthy person's forehead is 26° C. when the ambient temperature is 12° C., that the surface temperature of a healthy person's forehead is 28° C. when the ambient temperature is 18° C., that the surface temperature of a healthy person's forehead is 30° C. when the ambient temperature is 24° C., that the surface temperature of a healthy person's forehead is 32° C. when the ambient temperature is 30° C., and that the surface temperature of a healthy person's forehead is 34° C. when the ambient temperature is 36° C.

In the temperature-gauging section 612, ambient temperature that is the same as that of the wearer is measured. In the image-generating section 611, an image is generated on the basis of temperature gauged in the temperature-gauging section 612. The generated image is rendered so as to be presumed color-producing/color-changing status of 510 to 514 on the ambient thermochromic strip 51 in FIG. 9, configured utilizing a thermal indicator as follows. In FIGS. 9, 510 to 514 on the ambient thermochromic strip 51 are arranged to exhibit color production in/color change to red, green, or blue in temperature ranges (6° C. temperature spreads) of 8° C. to 14° C., 14° C. to 20° C., 20° C. to 26° C., 26° C. to 32° C., and 32° C. to 38° C. Generated in the image-generating section 611 is an image that reproduces the state of color-production/color-change to black, red, green, or blue of 510 to 514 on the ambient thermochromic strip 51, at the ambient temperature from the temperature-gauging section 612. The display section 610 displays the image from the image-generating section 611.

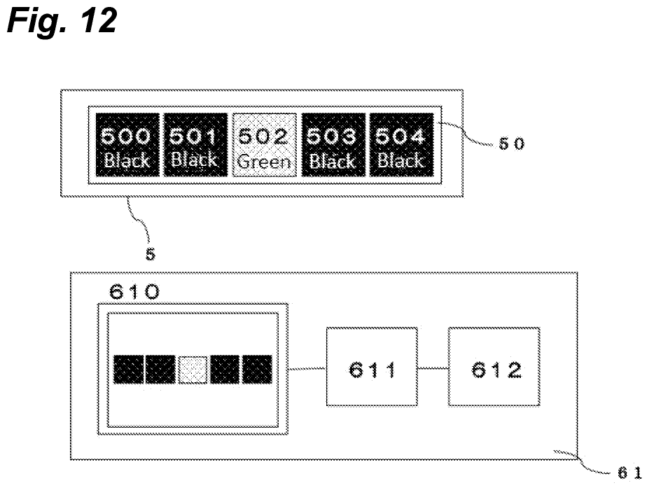

FIG. 12 is one example in which in Embodiment Example 5 color-producing/color-changing status that is observed in actuality has been schematically expressed. In this case, because the producing-color/changing-color state of the body-temperature thermochromic strip 50 and the producing-color/changing-color state of the ambient thermochromic strip 61 are about the same (the center color-producing in green), the wearer can be diagnosed as not having a fever. Specifically, with it being the case that an instance in which the ambient temperature is 24° C. and the surface temperature of the wearer's forehead is 30° C. is presented, because the strips have been designed, as the result of observation in advance, to cover an instance in which “the surface temperature of a healthy person's forehead is 30° C. when the ambient temperature is 24° C.,” the instance in FIG. 12 is an example in which the body-temperature state of a healthy person is displayed.

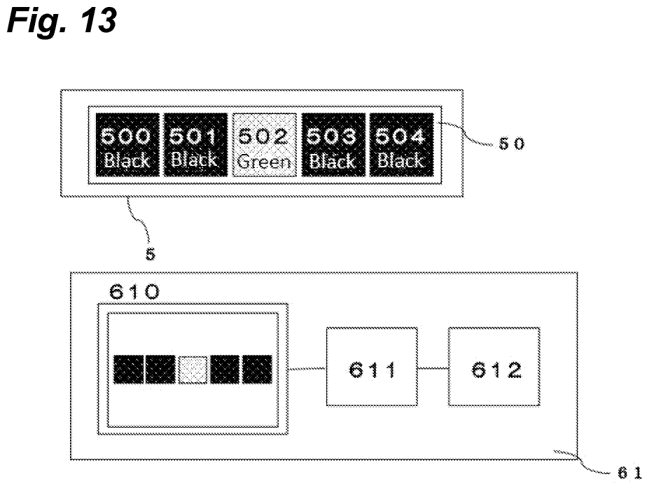

FIG. 13 is a separate example in which in Embodiment Example 5 color-producing/color-changing status that is observed in actuality has been schematically expressed. In this case, because the location in the body-temperature thermochromic strip 50 where producing-color/changing-color is exhibited is an area where the temperature is higher than (rightward of) the location in the ambient thermochromic strip 61 where producing-color/changing-color is exhibited, the wearer can be diagnosed as having a fever. Specifically, with it being the case that an instance in which the ambient temperature is 24° C. and the surface temperature of the wearer's forehead is 32° C. is presented, because the strips have been designed, as the result of observation in advance, to cover an instance in which “the surface temperature of a healthy person's forehead is 30° C. when the ambient temperature is 24° C.,” the instance in FIG. 13 is an example in which, with the surface temperature of the wearer's forehead being observed to be 2° C. higher than a healthy person's, the body-temperature state of a person having a fever is displayed.

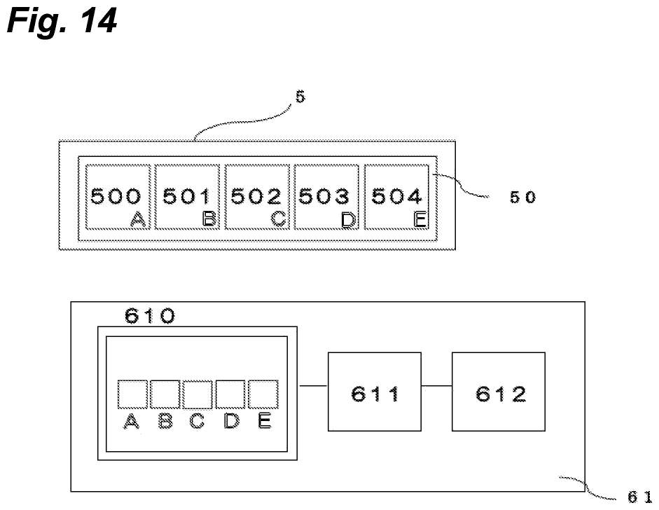

From comparing, as in FIGS. 12 and 13, the location in the body-temperature thermochromic strip 50 where producing-color/changing-color is exhibited and the location in the ambient thermochromic strip 61 where producing-color/changing-color is exhibited: An implementation in which they are segregated and in separated places makes them prone to being mistakenly read. They can be rendered to display, as in FIG. 14, with labeling A, B, C, D and E put on the respective blocks such as will indicate the locations where color is produced/color changes, configuring them so as to facilitate comparison of locations. The labeling may be alphabetical as in FIG. 14, or numbers can be utilized.

In Embodiment Example 5, as the thermal indicator cholesteric liquid crystals were taken up, but as long as it is something that will produce color/change color according to designated temperature it can be adopted.

Furthermore, likewise as with Embodiment Example 4, the respective blocks in the body-temperature thermochromic strip 50 and in the output image from the image-generating section 611 are landscape-oriented, but arranging them in circular form as in FIG. 10 is also possible.

While the embodiment examples of fever-diagnostic labels and fever-diagnostic masks involving the present invention have been described in the foregoing, it will be understood that various modifications are possible without departing from the technical scope of the present invention.

### INDUSTRIAL EXPLOITABILITY

According to a fever-diagnostic label or a fever-diagnostic mask involving the present invention, since change in thermochromic strips on the fever-diagnostic label or the fever-diagnostic mask enable visually, readily determining diagnosis of fever in a wearer, exploiting them broadly is possible not only in situations in which large numbers of people are subject to testing—in schools, hospitals and other medical facilities, and administrative organizations—but also for household use in order to respond to concerns over the spread of infectious diseases.

### DESCRIPTION OF REFERENCE MARKS

1: fever-diagnostic label
10: body-temperature thermochromic strip
11: ambient thermochromic strip
115: heat insulator
2: fever-diagnostic mask
20: mask body
21: support cord
22: thermochromic strip
3: fever-diagnostic mask
30: mask body
31, 41: support cords
32, 42: thermochromic strips
4: fever-diagnostic label
50: body-temperature thermochromic strip
51: ambient thermochromic strip

1. A fever-diagnostic label as well as a fever-diagnostic mask provided with:
a body-temperature thermochromic strip that is responsive to body temperature of a wearer; and
an ambient thermochromic strip that is responsive to ambient temperature; wherein
body-temperature state of the wearer is diagnosed by comparing color change in the body-temperature thermochromic strip with color change in the ambient thermochromic strip.

2. The fever-diagnostic label as well as fever-diagnostic mask set forth in claim 1, characterized in being coated thereon with temperature-dependent color-changing ink or pigment to form the body-temperature thermochromic strip and the ambient thermochromic strip.

3. The fever-diagnostic label as well as fever-diagnostic mask set forth in claim 1, characterized in being fitted thereon with a sticker printed with ink or pigment to form the body-temperature thermochromic strip and the ambient thermochromic strip.

4. The fever-diagnostic label as well as fever-diagnostic mask set forth in claim 1, characterized in being fitted thereon with a thermochromic film employing heat-sensitive liquid crystals to form the body-temperature thermochromic strip and the ambient thermochromic strip.

5. The fever-diagnostic mask set forth in any of claims 1 through 4, characterized in that a mask body of the fever-diagnostic mask is perforated with a desired one or plurality of holes in location(s) on the body-temperature thermochromic strip.

6. The fever-diagnostic label as well as fever-diagnostic mask set forth in any of claims 1 through 5, characterized in that:
the ambient thermochromic strip is segregated from the thermochromic strip on the fever-diagnostic label as well as fever-diagnostic mask; and
the body-temperature thermochromic strip is furnished on the thermochromic strip on the fever-diagnostic label or fever-diagnostic mask, with the ambient thermochromic strip being furnished in a position where it may be sight-checked by an observer under the same ambient temperature as the wearer of the fever-diagnostic label or fever-diagnostic mask.

7. The fever-diagnostic label as well as fever-diagnostic mask set forth in claim 6, characterized in that the ambient thermochromic strip is furnished with:
a temperature-gauging section for gauging the ambient temperature;
an image-generating section for generating an image caused to produce color/change color according to ambient temperature; and
a display section for displaying an image output from the image-generating section.

8. The fever-diagnostic label as well as fever-diagnostic mask set forth in any of claims 1 through 7, characterized in that a labelling display is made on the body-temperature thermochromic strip and the ambient thermochromic strip for indicating positions where color is produced or color is changed.

9. A fever-diagnosing method wherein, in a fever-diagnostic label as well as a fever-diagnostic mask provided with a body-temperature thermochromic strip that is responsive to the body temperature of a wearer and an ambient thermochromic strip that is responsive to ambient temperature:
the body-temperature thermochromic strip and the ambient thermochromic strip are formed by coating on ink or pigment that changes color depending on temperature or else by fitting them on as a sticker, or otherwise are formed by fitting on a thermochromic film employing heat-sensitive liquid crystals; and
the body-temperature state of the wearer is diagnosed by comparing color change in the body-temperature thermochromic strip with color change in the ambient thermochromic strip.
